(19) United States
(12) Patent Application Publication
(10) Pub. No.: US 2025/0268760 A1
(43) Pub. Date: Aug. 28, 2025

(54) STRETCH TO FIT BANDAGE

(71) Applicant: 3M INNOVATIVE PROPERTIES COMPANY, St. Paul, MN (US)

(72) Inventors: Dale R. Wiehe, Lake Elmo, MN (US); Diane L. Emslander, Grant, MN (US); Amy J. Krohn, Oakdale, MN (US)

(21) Appl. No.: 18/857,364

(22) PCT Filed: Apr. 13, 2023
(86) PCT No.: PCT/IB2023/053798

§ 371 Date: Oct. 16, 2024

Related U.S. Application Data
(60) Provisional application No. 63/363,312, filed on Apr. 20, 2022.

(51) Int. Cl. A61F 13/0203; A61F 13/00

(52) U.S. Cl. CPC A61F 13/0203; A61F 13/0008; A61F 2013/00119

(57) ABSTRACT

The present invention is a stretch to fit bandage having a stretchable backing layer and an adhesive layer positioned adjacent at least a portion of the backing layer. The backing layer has a percent elongation of at least about 200% and an elastic recovery after elongation of less than about 35%.

### FIELD OF THE INVENTION

The present invention is related generally to the field of bandages. In particular, the present invention is a stretch to fit bandage.

### BACKGROUND

Medical adhesive bandages typically include a backing, an absorbent pad, and a pressure sensitive adhesive to maintain the medical adhesive bandage in place. As referred to below, “bandage” or “bandages” will be understood to refer to medical adhesive bandages and/or dressings. The bandages are typically used to cover cuts, scrapes, and other skin conditions. They are typically packaged individually to maintain cleanliness of the bandages before use. As an added benefit, the individual packaging also provides the opportunity to provide bandages that are sterile until removed from the packaging. Typically, however, sterility of the bandages is only a secondary concern to the primary requirement that the bandages be clean and free of foreign matter before application.

Most bandages have a fixed size. This can be problematic if a wound is larger than the bandage, as the user must then obtain a larger bandage or open a number of individually packaged bandages to cover the wound. At the opposite end of the size problem is that the wound may be significantly smaller than bandages available to the user and, as a result, use of the bandage may be wasteful because a smaller bandage would be sufficient to cover the wound.

A currently available method of offering user-sized bandages involves providing a roll-form bandage having a continuous backing and continuous length absorbent pad. The user removes the roll from a box or other storage device, cuts a bandage of the desired length from the roll and replaces the roll in the box. In addition to providing opportunities for contamination of the roll if improperly handled, this delivery system requires the user to obtain and use scissors or another cutting instrument to separate a bandage from the roll. This delivery system also leaves two edges of the pad without an adhesive border to seal to the skin to keep out dirt and germs.

Another attempt at solving the fixed size issue involves providing an approximately 10 centimeter long backing and associated pad in sheet form that is packaged in a box or envelope. To obtain a bandage having the desired size, the user removes the sheet bandage from the package, cuts off a piece having the desired size, and replaces the remaining portion of the sheet bandage back in the package. Like the bandages dispensed from a roll, the handling and storage of the larger sheet bandage provides opportunities for contamination if improperly handled and also requires the user to obtain and use scissors or other cutting device to separate the desired bandage from the sheet.

Yet another current solution involves providing a foam tape perforated at pre-determined intervals across the tape and placing absorbent pads on the tape between the perforations to provide bandages that can be individually dispensed by separating the tape along the lines of perforation. This material is also provided in roll form in a box or other storage device. To dispense a bandage, the user removes the roll from the box or other storage device, cuts or tears along a line of perforations a bandage of the desired length from the roll and replaces the roll in the box. As discussed above, the handling and storage of bandages in roll-form provides opportunities for contamination of the remaining material because the pads have been exposed both during storage and dispensing. Another problem with this delivery system is the difficulty in accurately placing the pads between the lines of perforation. If the pads cross the perforation lines, it is difficult to tear the tape along the lines of perforation and, in addition, the pad edges can fray.

### SUMMARY

In one embodiment, the present invention is a stretch to fit bandage including a stretchable backing layer and an adhesive layer positioned adjacent at least a portion of the backing layer. The backing layer has a percent elongation of at least about 200% and an elastic recovery after elongation of less than about 35%.

### BRIEF DESCRIPTION OF THE DRAWINGS

These figures are not drawn to scale and are intended merely for illustrative purposes. The disclosure may be more completely understood in consideration of the following detailed description of various embodiments of the disclosure in connection with the accompanying drawings, in which:

FIG. 1 is a cross-sectional view of an embodiment of a bandage of the present invention.

FIG. 2 is a top view of the bandage of the present invention being stretched in multiple directions.

FIG. 3 is a top view of an embodiment of a release liner of the present invention.

FIG. 4 is a top view of a first embodiment of a release liner system of the present invention.

FIG. 5 is a top view of a second embodiment of a release liner system of the present invention.

In the following detailed description, reference may be made to the accompanying set of drawings that form a part hereof and in which are shown by way of illustration several specific embodiments. It is to be understood that other embodiments are contemplated and may be made without departing from the scope or spirit of the present disclosure.

### DETAILED DESCRIPTION

The present invention is a bandage that is stretchable with little to no elastic recovery or rebound after strain, also referred to as a dead stretch. The bandage can thus be stretched from its initial size to a selected size. The selected size can depend, for example, on the size of the wound or the body part to be covered. One advantage of the bandage of the present invention is its capability to stretch and conform to various body parts, such as knees, elbows, and fingers. The stretchable bandage of the present invention thus offers a versatile bandage of varying sizes. In addition, the bandage also resolves the need for assorted size packages and avoids bandages that rarely get used due to being the wrong size.

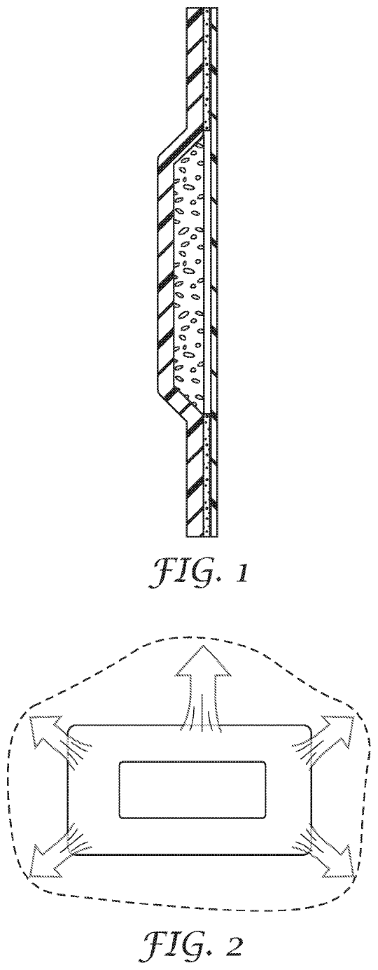

FIG. 1 shows cross-sectional view of an embodiment of the bandage of the present invention. The bandage generally includes a backing layer and an adhesive layer positioned on a first surface of the backing layer. The bandage may also optionally include an absorbent pad and/or a release liner. When included, the absorbent pad is attached to the first surface of the backing layer. Typically, the absorbent pad will be attached to the backing layer using the adhesive layer, but it may alternatively be attached to the backing layer by any suitable method. The release liner then covers at least a portion of the exposed adhesive layer. Each layer will be discussed in further detail below.

The backing layer may be a single layer film, a laminate of films or a multilayer film. The film construction of the backing layer is such that it is able to be manually expanded to, for example, fit a wound shape or size, and then hold the new shape (FIG. 2). In one embodiment, the backing layer has a strain, or percent elongation, of at least about 200%, at least about 250%, at least about 300%, at least about 350%, at least about 400%, at least about 450%, at least about 500%, at least about 550%, at least about 600%, at least about 650%, at least about 700%, at least about 750%, at least about 800%, at least about 900%, and at least about 1000%. Tensile and percent elongation can be measured, for example, using ASTM-D3759. The relaxation property, or elastic recovery after strain/elongation, of the backing layer is minimal to zero in order for the bandage to retain the shape to which it is stretched. In addition, minimal elastic recovery also minimizes or prevents tension blisters caused by any residual relaxation. In one embodiment, the backing layer has an elastic recovery of less than about 35%, less than about 30%, less than about 25%, less than about 20%, less than about 15%, less than about 10%, and less than about 5% after elongation. In one embodiment, the percent elastic recovery can be measured using an Instron 5944, available from Instron located at Norwood, MA. A 1″×8″ die is used on a lab clicker press to cut out samples. The test sample dimensions are 1″(w)×2″(l) so these 8″ samples can be cut into 2×4″ samples. The samples are equilibrated in a CTH (controlled temperature and humidity) room for at least 8 hours (at 70° F./50% rH). A 2 kNB load cell and jaws with 1″ wide rubber-faced grips are used on the Instron. The jaw/grip gap is set at 2″ and the speed is a 10″/min. pull rate. The Instron is calibrated and the load and extension are zeroed. Each end of the test sample is clamped in the Instron grips and the test method sequence is as follows: ramp to 100% elongation (i.e. from 2″ to 4″), hold for 1 minute, un-ramp to 0% elongation, hold for 3 minutes, re-ramp to 100% elongation, and return. The percent elastic recovery is then documented.

In one embodiment, the backing layer is capable of stretching in at least one direction, such as width or length. Particularly, the backing layer is capable of stretching in at least four directions, and more particularly, the backing layer is capable of stretching along any and all given axes. The stretchability of the backing layer can result from various factors, including for example, the material of the backing layer and the formation of openings, such as slits or holes, in the backing layer. Slitting the backing layer can allow for expansion of the backing layer. The slits may be formed in a pattern or may be random. In some embodiments, the pattern of slits can be formed onto a core layer of the stretchable backing layer, which can then be laminated with one or more continuous polymeric films or foams. In one embodiment, the backing layer is at least partially permeable to moisture vapor released through the user's skin. In some instances, permeability can be obtained and/or increased by providing a number of openings in the backing layer. Examples of suitable backing layer materials include, but are not limited to: one or more polymeric foams, one or more polymeric films, various viscoelastic materials, stiff materials, and combinations thereof. Examples of suitable backing layer film materials include polyethylene copolymers and polyurethanes. Examples of commercially suitable backing layer materials include, but are not limited to: Affinity 8852, Affinity 1850, and Infuse 9507, available from Dow Chemical located at Midland, MI; Optima TC120, available from ExxonMobil located at Irving, TX; Kraton 1657, available from Kraton Corporation located at Houston, TX; Estane 58245 and MVT 75AT3, available from The Lubrizol Corporation located at Wickliffe, OH; and PEBAX® MV 1074, available from Arkema located at Colombes, France, and combinations thereof. In one embodiment, the backing layer may have a thickness of, for example, up to about 2 mils and particularly up to about 5 mils.

The adhesive layer used in the present invention is made of an adhesive known to those of skill in the art which can be applied to the skin. In one example, the adhesive layer is a pressure sensitive adhesive. A general description of useful pressure sensitive adhesives may be found in the Encyclopedia of Polymer Science and Engineering, Vol. 13, Wiley-Interscience Publishers (New York, 1988). An additional description of useful pressure-sensitive adhesives may be found in the Encyclopedia of Polymer Science and Technology, Vol. 1, Interscience Publishers (New York, 1964). Any suitable composition, material or ingredient can be used in the pressure sensitive adhesive Exemplary pressure sensitive adhesives utilize one or more thermoplastic elastomers, e.g., in combination with one or more tackifying resins. In some embodiments, the adhesive is not a pressure sensitive adhesive.

Examples of suitable adhesives can include at least one of rubber, silicone, or acrylic based adhesives. In some embodiments, the adhesive can include tackified rubber adhesives, such as natural rubber; olefins; silicones, such as silicone polyureas or silicone block copolymers; synthetic rubber adhesives such as polyisoprene, polybutadiene, and styrene-isoprene-styrene, styrene-ethylene-butylene-styrene and styrene-butadiene-styrene block copolymers, and other synthetic elastomers; and tackified or untackified acrylic adhesives such as copolymers of isooctylacrylate and acrylic acid, which can be polymerized by radiation, solution, suspension, or emulsion techniques; polyurethanes; silicone block copolymers; and combinations of the above.

Nonlimiting examples of useful adhesives include those described in U.S. Pat. No. 4,112,177 (particularly the tackified acrylate “skin layer adhesive” described in Example 1). Other suitable adhesives are described in U.S. Pat. No. 5,648,166. Additional suitable adhesives include the acrylate copolymers described in U.S. Pat. No. RE 24,906. Another example of a useful adhesive is an 70:15:15 isooctyl acrylate:ethyleneoxide acrylate:acrylic acid terpolymer, as described in U.S. Pat. No. 4,737,410 (see Example 31). Other useful adhesives are described in U.S. Pat. Nos. 3,389,827, 4,112,213, 4,310,509, and 4,323,557. While the application lists particular adhesives, any adhesive safe for application onto human or animal skin can be used without departing from the intended scope of the present invention. In one embodiment, the adhesive layer is formed of material that is of the class known as “hypoallergenic” adhesives.

Inclusion of medicaments or antimicrobial agents in the adhesive is also contemplated, as described in U.S. Pat. Nos. 4,310,509 and 4,323,557. Generally, any known additives useful in the formulation of adhesives may also be included. Additives include plasticizers, anti-aging agents, ultraviolet stabilizers, colorants, thermal stabilizers, anti-infective agents, fillers, crosslinkers, as well as mixtures and combinations thereof. In certain embodiments, the adhesive can be reinforced with fibers or a fiber scrim which may include inorganic and/or organic fibers. Suitable fiber scrims may include woven, non-woven or knit webs or scrims. For example, the fibers in the scrim may include wire, ceramic fiber, glass fiber (for example, fiberglass), and organic fibers (for example, natural and/or synthetic organic fibers).

In one embodiment, the adhesive layer allows the bandage to have a hold time to human skin of at least about 8 hours, particularly at least about 24 hours, and more particularly at least about 48 hours. Hold time can be determined by controlled human clinical trials. Samples are applied to a clean surface and monitored over time for adhesion, edge lift, and functionality. Biostatistics are used to establish end points.

The bandage of the present invention may transmit moisture vapor to increase user comfort. While moisture vapor transmission can be achieved through the selection of an appropriate adhesive, it is also contemplated in the present invention that other methods of achieving a high relative rate of moisture vapor transmission may be used, such as pattern coating the adhesive on the backing layer, as described in U.S. Pat. No. 4,595,001.

When a pad is included in the bandage, the pad is an absorbent pad. In one embodiment, the pad is also made of a stretchable material. The stretchability of the pad can result from various factors, including for example, the material of the pad and/or the formation of openings, such as slits or holes, in the pad. In one embodiment, the pad is skip slit utilizing slitting technology to allow for expansion when the bandage is stretched. Other methods of slitting using various patterns to allow for a 2-way stretch, a 4-way stretch, to an all-way stretch may also be used. In one embodiment, the pad is centered between the edges of the backing layer. The pad can be manufactured from a number of materials including, but not limited to: woven cloth or nonwoven, rayon, fibers, particles, hydrocolloids, foams, and combinations thereof. The pad may also contain a number of substances, including antimicrobial agents, anesthetics, anti-itch agents, drugs for transdermal drug delivery, chemical indicators to monitor hormones or other substances in a patient, combinations thereof and the like. Furthermore, although the pad is shown as being generally rectangular and centered on the backing layer, the pad can take any appropriate shape and/or be located off-center on the backing layer without departing from the intended scope of the present invention.

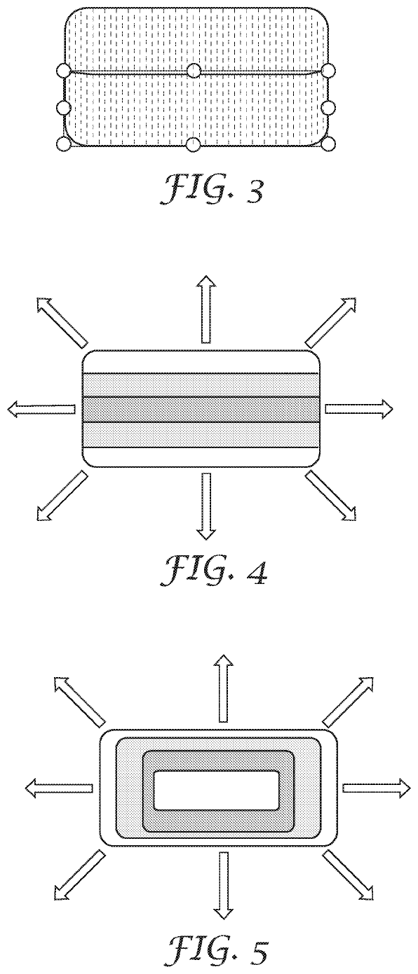

When included in the bandage of the present invention, the release liner covers at least a portion of the adhesive layer opposite the backing layer. The release liner provides a release surface from which the bandage can be removed for placement onto a wound site. The release liner may be made from a material available from a variety of manufacturers in a wide variety of proprietary formulations. In one embodiment, the release liner is also stretchable with minimal to zero elastic recovery after strain. The stretchability of the release liner can result from various factors, including for example, the material of the release liner and the formation of openings, such as slits or holes, in the release liner. In one embodiment, the release liner is skip slit utilizing slitting technology to allow for expansion when the bandage is stretched. An embodiment of a slitted release liner is shown in FIG. 3. Stretching the bandage while the release liner is still in place allows for maximum adhesion. If the release liner is removed prior to stretching, contact with the adhesive layer may leave dust and debris, such as skin cells and/or skin oils, on the adhesive layer, reducing adhesion. Various configurations of the release liner can be used to allow for stretching of the backing layer while the release liner is still attached.

In one configuration shown in FIG. 4, a first release liner is positioned along a first edge of the bandage and a second release liner is positioned along a second, opposite edge of the bandage. A third release liner is then positioned on the adhesive layer still exposed along the center of the bandage, overlapping the first and second liners on the respective edges of the third liner. These areas of overlap allow the backing layer to slide and stretch.

In another configuration shown in FIG. 5, a first release liner has a center hole cut into it and a second release liner that covers the center so as to allow slip to occur during stretching of the backing layer. There must be some exposed adhesive layer around the pad so that the second release liner can attach to the exposed adhesive, cover the pad, and overlap the first release liner.

In a third configuration, the bandage includes a first and a second release liner on two opposing edges of the bandage and a third center release liner that remains on the package. In practice, as the bandage is peeled off from the packaging, the center area is exposed and the first and second release liners protecting the two edges become paddles for holding the bandage and conducting the stretch. Alternatively, the release liner can be in the shape of a ring that is lifted out of the packaging, exposing a complete open center to facilitate the stretch. Additionally, a release liner frame on the outer edges of the bandage can be used to stabilize the bandage and the entire release liner stays within the packaging. In one embodiment, additional sections of the release liner frame may be needed to allow for the stretch.

Those skilled in the art will normally test liner materials in simulated use conditions against an adhesive of choice to arrive at a product with the desired release characteristics. In one embodiment, the release liner does not need to be stretchable and is removed prior to the backing layer being stretched. Release liners that are suitable for use with the bandage of the present invention can be made of, for example: kraft papers, polyethylene, polypropylene, polyester or composites of any of these materials. In one embodiment, the release liner includes release agents such as fluorochemicals or silicones. For example, U.S. Pat. No. 4,472,480, the disclosure of which is hereby incorporated by reference, describes low surface energy perfluorochemical liners. Some suitable release liners include papers, polyolefin films, or polyester films coated with silicone release materials. Examples of commercially available silicone coated release papers SILOX-coated release papers, available from the Akrosil Division of International Paper (Menasha, Wis.), and the silicone release papers supplied by Daubert Coated Products, Inc. (Willowbrook, Ill.). An example of a suitable release liner includes, but is not limited to, a co-extruded PLA/LDPE with a Bynel 22E780 tie layer.

The stretch to fit bandage of the present invention allows for a bandage that can fit a large variety of sizes of wounds. This allows for one product to fit most wounds and remove the need for multiple size bandages/dressings or the need to estimate which size bandage is best for a particular wound. In addition, a slitted pad may also increase the rate of absorbency as more surface area is exposed to the wound.

What is claimed is:

1. A stretch to fit bandage comprising:
a stretchable backing layer; and
an adhesive layer positioned adjacent at least a portion of the backing layer,
wherein the backing layer has a percent elongation of at least about 200%, and
wherein the backing layer has an elastic recovery after elongation of less than about 35%.

2. The stretch to fit bandage of claim 1, wherein the adhesive layer is comprised of one of rubber, an acrylic, and a silicone.

3. The stretch to fit bandage of claim 1, wherein the backing layer has a percent elongation of at least about 500%.

4. The stretch to fit bandage of claim 1, wherein the backing layer has a percent elongation of at least about 800%.

5. The stretch to fit bandage of claim 1, wherein the bandage has an elastic recovery after elongation of less than about 25%.

6. The stretch to fit bandage of claim 1, wherein the bandage has an elastic recovery after elongation of less than about 15%.

7. The stretch to fit bandage of claim 1, wherein the bandage is capable of stretching along any given axis.

8. The stretch to fit bandage of claim 1, wherein the bandage has a hold time of at least about 8 hours.

9. The stretch to fit bandage of claim 1, wherein the bandage has a hold time of at least about 24 hours.

10. The stretch to fit bandage of claim 1, wherein the bandage has a hold time of at least about 48 hours.

11. The stretch to fit bandage of claim 1, further comprising an absorbent element positioned adjacent the backing layer.

12. The stretch to fit bandage of claim 11, wherein the absorbent element may comprise one of a: woven or nonwoven cloth, rayon, nonwovens, fibers, particles, hydrocolloids, foams, and combinations thereof.

13. The stretch to fit bandage of claim 11, wherein the absorbent element is a pad.

14. The stretch to fit bandage of claim 1, wherein the backing layer is stretchable in at least four directions.

15. The stretch to fit bandage of claim 1, further comprising a release liner positioned adjacent the adhesive layer.

16. The stretch to fit bandage of claim 15, wherein the release liner is stretchable.
